(19) United States
(12) Patent Application Publication
(10) Pub. No.: US 2025/0001194 A1
(43) Pub. Date: Jan. 2, 2025

(54) Treating Liquid Tumors and Using an MRI Scanner to Both Image a Tumor and Treat the Tumor Using Tumor Treating Fields (TTFields)

(71) Applicant: Novocure GmbH, Root D4 (CH)

(72) Inventor: Kristen W. Carlson, Concord, MA (US)

(73) Assignee: Novocure GmbH, Root D4 (CH)

(21) Appl. No.: 18/756,904

(22) Filed: Jun. 27, 2024

Related U.S. Application Data
(60) Provisional application No. 63/571,654, filed on Mar. 29, 2024.
(60) Provisional application No. 63/524,118, filed on Jun. 29, 2023.

(51) Int. Cl. A61N 2/02; A61B 5/00; A61B 5/055; A61N 2/00

(52) U.S. Cl. CPC A61N 2/02; A61B 5/0036; A61B 5/055; A61N 2/004

(57) ABSTRACT

A multi-mode MRI scanner relies on conventional hardware and control software to operates in a typical first mode that captures images of whatever body part is positioned within the bore of the scanner. The multi-mode MRI scanner also operates in a novel second mode in which (a) a first subset of the MRI scanner's electromagnetic coils is driven with an AC current (which induces a first alternating magnetic field in the body part), and (b) a second subset of the MRI scanner's electromagnetic coils is driven with an AC current (which induces a second alternating magnetic field with a different direction in the body part). Steps (a) and (b) are repeated in an alternating sequence. The first and second alternating magnetic fields give rise to corresponding alternating electric fields in two different directions, which provide a therapeutic effect (e.g., for treating a tumor) in the body part.

### CROSS REFERENCE TO RELATED APPLICATIONS

This application claims the benefit of US Provisional Application 63/524,118 filed Jun. 29, 2023, and U.S. Provisional Application 63/571,654, filed Mar. 29, 2024, each of which is incorporated herein by reference in its entirety.

### BACKGROUND

Tumor Treating Fields (TTFields) therapy is a proven approach for treating tumors using alternating electric fields at frequencies e.g., between 5 kHz-5 MHz, more commonly 100-300 KHz. The alternating electric fields are induced by electrode assemblies (e.g., arrays of capacitively coupled electrodes, also called transducer arrays) placed on the subject's skin on opposite sides of the subject's body. More specifically, when an AC voltage is applied between opposing electrode assemblies, an AC current is coupled through the electrode assemblies and into the subject's body, which induces the alternating electric field in the target region. Notably, TTFields are only effective at treating tumors when the intensity of the alternating electric field is on the order of 1 V/cm or higher.

Various tumor locations can present obstacles to the delivery TTFields using the conventional approach of positioning electrode assemblies on the subject's body. Tumors in the electrically resistive spinal column, and in bone-encased blood marrow are examples. Furthermore, TTFields have typically been used to treat localized, solid tumors, and have heretofore been unable to treat widely-dispersed tumors such as blood cancers or solid tumors that are widely dispersed (e.g., in lymph nodes in disparate locations in the body). Currently these widely dispersed tumors cannot be, and are not, treated with a single TTFields application. Consecutive TTFields applications would be necessary, moving the surface electrodes to target different locations of the tumor. But while TTFields are treating the tumor in one location, it will be growing or metastasizing at the other locations.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier so that, e.g., chemotherapy drugs can reach the brain.

### SUMMARY OF THE INVENTION

One aspect of the invention is directed to a first method of treating a body part positioned within a bore of an MRI scanner using alternating magnetic fields. The MRI scanner has a plurality of electromagnetic coils. The first method comprises (a) driving a first subset of the electromagnetic coils with an AC current at a frequency between 5 kHz and 1 MHz for a first interval of time so that a first alternating magnetic field is induced in the body part; (b) driving a second subset of the electromagnetic coils with an AC current at a frequency between 5 kHz and 1 MHz for a second interval of time so that a second alternating magnetic field is induced in the body part; and (c) repeating steps (a) and (b) in an alternating sequence a plurality of times. The first alternating magnetic field and the second alternating magnetic field have different directions, a sum of all the first intervals of time is at least one hour, and a sum of all the second intervals of time is at least one hour.

In some instances of the first method, the electromagnetic coils are radiofrequency transmitter coils. In some instances of the first method, the electromagnetic coils are gradient coils. In some instances of the first method, the electromagnetic coils are surface coils.

In some instances of the first method, the first subset of the electromagnetic coils is driven with an AC current at a frequency between 100 kHz and 300 kHz, and the second subset of the electromagnetic coils is driven with an AC current at a frequency between 100 kHz and 300 kHz.

In some instances of the first method, each of the first intervals of time and each of the second intervals of time is between 25 ms and 1800 s. In some instances of the first method, each of the first intervals of time and each of the second intervals of time is between 0.2 s and 60 s. In some instances of the first method, steps (a) and (b) are repeated in an alternating sequence at least 100 times.

Some instances of the first method further comprise using the MRI scanner to capture an image of a tumor in the body part while the body part remains positioned within the bore of the MRI scanner.

In some instances of the first method, portions of the MRI scanner that are responsible for generating a static magnetic field remain active while steps (a) and (b) are implemented. In some instances of the first method, portions of the MRI scanner that are responsible for generating a static magnetic field remain deactivated while steps (a) and (b) are implemented.

Another aspect of the invention is directed to a first MRI scanner that comprises an electromagnet, a plurality of electromagnetic coils including a set of radiofrequency transmitter and receiver coils, a plurality of gradient coils, and, optionally, a plurality of surface coils; and a controller. The electromagnet is configured to generate a static magnetic field within a bore. The set of radiofrequency transmitter and receiver coils are configured to generate and receive electromagnetic fields within the static magnetic field. The plurality of gradient coils are configured to introduce magnetic gradients superimposed upon the static magnetic field. The controller is configured to operate the MRI scanner in a first mode in which images of a body part positioned within the bore are obtained. And the controller is further configured to operate the MRI scanner in a second mode in which (a) a first subset of the electromagnetic coils is driven with an AC current at a frequency between 5 kHz and 1 MHz for a first interval of time so that a first alternating magnetic field is induced in the body part, and (b) a second subset of the electromagnetic coils is driven with an AC current at a frequency between 5 kHz and 1 MHz for a second interval of time so that a second alternating magnetic field is induced in the body part. Steps (a) and (b) are repeated in an alternating sequence a plurality of times. The first alternating magnetic field and the second alternating magnetic field have different directions. A sum of all the first intervals of time is at least one hour, and a sum of all the second intervals of time is at least one hour.

In some embodiments of the first MRI scanner, the electromagnetic coils that are driven with the AC current are radiofrequency transmitter coils. In some embodiments of the first MRI scanner, the electromagnetic coils that are driven with the AC current are gradient coils. In some embodiments of the first MRI scanner, the electromagnetic coils that are driven with the AC current are surface coils.

In some embodiments of the first MRI scanner, the first subset of the electromagnetic coils is driven with an AC current at a frequency between 100 kHz and 300 kHz, and the second subset of the electromagnetic coils is driven with an AC current at a frequency between 100 kHz and 300 kHz.

In some embodiments of the first MRI scanner, each of the first intervals of time and each of the second intervals of time is between 25 ms and 1800 s. In some embodiments of the first MRI scanner, each of the first intervals of time and each of the second intervals of time is between 0.2 s and 60 s. In some embodiments of the first MRI scanner, steps (a) and (b) are repeated in an alternating sequence at least 100 times.

In some embodiments of the first MRI scanner, the electromagnet configured to generate the static magnetic field within the bore remains active while the MRI scanner is operating in the second mode. In some embodiments of the first MRI scanner, the electromagnet configured to generate the static magnetic field within the bore remains deactivated while the MRI scanner is operating in the second mode.

Another aspect of the invention is directed to a second MRI scanner that comprises an electromagnet, a plurality of electromagnetic coils including a set of radiofrequency transmitter and receiver coils, a plurality of gradient coils, and, optionally, a plurality of surface coils; and a controller. The electromagnet is configured to generate a static magnetic field within a bore. The set of radiofrequency transmitter and receiver coils is configured to generate and receive electromagnetic fields within the static magnetic field. The plurality of gradient coils is configured to introduce magnetic gradients superimposed upon the static magnetic field. The controller is configured to operate the MRI scanner in a first mode in which images of a body part positioned within the bore are obtained. And the controller is further configured to operate the MRI scanner in a second mode in which a subset of the electromagnetic coils is driven with an AC current at a frequency between 5 kHz and 1 MHz so that an alternating magnetic field is induced in the body part for at least four hours.

In some embodiments of the second MRI scanner, the electromagnetic coils that are driven with the AC current are radiofrequency transmitter coils. In some embodiments of the second MRI scanner, the electromagnetic coils that are driven with the AC current are gradient coils. In some embodiments of the second MRI scanner, the electromagnetic coils that are driven with the AC current are surface coils.

In some embodiments of the second MRI scanner, the subset of the electromagnetic coils is driven with an AC current at a frequency between 100 kHz and 300 kHz. In some embodiments of the second MRI scanner, the electromagnet configured to generate the static magnetic field within the bore remains active while the MRI scanner is operating in the second mode. In some embodiments of the second MRI scanner, the electromagnet configured to generate the static magnetic field within the bore remains deactivated while the MRI scanner is operating in the second mode.

Another aspect of the invention is directed to a second method of treating a liquid tumor in a subject's body that includes a volume of blood. The second method comprises applying an alternating magnetic field to the subject's body. The alternating magnetic field has a frequency between 50 kHz and 1 MHz, and the alternating magnetic field induces an alternating electric field having a strength of at least 0.1 V/cm in at least one-third of the volume of blood.

In some instances of the second method, the induced alternating electric field inhibits proliferation of liquid tumor cancer cells in the subject's body. In some instances of the second method, the induced alternating electric field reduces the number of liquid tumor cancer cells in the subject's body by at least 10%.

In some instances of the second method, the alternating magnetic field induces an alternating electric field having a strength of at least 1 V/cm in at least one-third of the volume of blood. Optionally, in these instances, the alternating magnetic field is applied to the subject's body for at least 12 hours per day, at least 5 days per week.

In some instances of the second method, the alternating magnetic field induces an alternating electric field having a strength of at least 1 V/cm in at least one-third of the volume of blood, the alternating magnetic field is applied to the subject's body for at least 12 hours per day, at least 5 days per week, and the induced alternating electric field has a strength of at least 1 V/cm in at least half of the subject's ribs, at least half of the subject's vertebrae, at least half of the subject's sternum, and at least half the subject's pelvic bones. Optionally, in these instances, the alternating magnetic field induces an alternating electric field having a strength of at least 1 V/cm in at least half the volume of blood.

In some instances of the second method, the alternating magnetic field induces an alternating electric field having a strength of at least 10 V/cm in at least one-third of the volume of blood. Optionally, in these instances, the alternating magnetic field is applied to the subject's body for at least 2 hours per day, at least 3 days per week.

In some instances of the second method, the alternating magnetic field induces an alternating electric field having a strength of at least 10 V/cm in at least one-third of the volume of blood, the alternating magnetic field is applied to the subject's body for at least 2 hours per day, at least 3 days per week, and the induced alternating electric field has a strength of at least 10 V/cm in at least half of the subject's ribs, at least half of the subject's vertebrae, at least half of the subject's sternum, and at least half the subject's pelvic bones. Optionally, in these instances, the alternating magnetic field induces an alternating electric field having a strength of at least 10 V/cm in at least half the volume of blood.

Another aspect of the invention is directed to a first apparatus for treating a liquid tumor in a subject's body that includes a volume of blood. The first apparatus comprises a platform, at least one alternating voltage source, and at least one electromagnetic coil. The at least one alternating voltage source has a frequency between 50 kHz and 1 MHz. The at least one electromagnetic coil is configured to generate at least one alternating magnetic field when driven by the at least one alternating voltage source. And the platform, the at least one alternating voltage source, and the at least one electromagnetic coil are configured and positioned such that when the subject's body is supported by the platform, the at least one alternating magnetic field generated by the at least one electromagnetic coil will induce an alternating electric field having a strength of at least 0.1 V/cm in at least one-third of the volume of blood.

In some embodiments of the first apparatus, the platform, the at least one alternating voltage source, and the at least one electromagnetic coil are configured and positioned such that when the subject's body is supported by the platform, the at least one alternating magnetic field generated by the at least one electromagnetic coil will induce an alternating electric field having a strength of at least 1 V/cm in at least half of the subject's ribs, at least half of the subject's vertebrae, at least half of the subject's sternum, and at least half the subject's pelvic bones. Optionally, in these embodiments, the platform, the at least one alternating voltage source, and the at least one electromagnetic coil are configured and positioned such that when the subject's body is supported by the platform, the at least one alternating magnetic field generated by the at least one electromagnetic coil will induce an alternating electric field having a strength of at least 1 V/cm in at least half the volume of blood.

In some embodiments of the first apparatus, the platform, the at least one alternating voltage source, and the at least one electromagnetic coil are configured and positioned such that when the subject's body is supported by the platform, the at least one alternating magnetic field generated by the at least one electromagnetic coil will induce an alternating electric field having a strength of at least 10 V/cm in at least one-third of the volume of blood. Optionally, in these embodiments, the platform, the alternating voltage source, and the electromagnetic coil are configured and positioned such that when the subject's body is supported by the platform, the at least one alternating magnetic field generated by the at least one electromagnetic coil will induce an alternating electric field having a strength of at least 10 V/cm in at least half of the subject's ribs, at least half of the subject's vertebrae, at least half of the subject's sternum, and at least half the subject's pelvic bones.

Optionally, in the embodiments described in the previous paragraph, the platform, the at least one alternating voltage source, and the at least one electromagnetic coil are configured and positioned such that when the subject's body is supported by the platform, the at least one alternating magnetic field generated by the at least one electromagnetic coil will induce an alternating electric field having a strength of at least 10 V/cm in at least half the volume of blood.

Another aspect of the invention is directed to a third method of treating a liquid tumor in a subject's body that includes a volume of blood. The third method comprises applying an alternating magnetic field to the subject's body. The alternating magnetic field has a frequency between 50 kHz and 1 MHZ, and the alternating magnetic field has a strength of at least 1 Tesla in at least one-third of the volume of blood. The alternating magnetic field is applied to the subject's body for at least 2 hours per day, at least 3 days per week.

In some instances of the third method, the alternating magnetic field induces an alternating electric field that inhibits proliferation of liquid tumor cancer cells in the subject's body. In some instances of the third method, the alternating magnetic field induces an alternating electric field that reduces the number of liquid tumor cancer cells in the subject's body by at least 10%.

In some instances of the third method, the alternating magnetic field has a strength of at least 1 Tesla in at least half of the subject's ribs, at least half of the subject's vertebrae, at least half of the subject's sternum, and at least half the subject's pelvic bones. Optionally, in these instances, the alternating magnetic field has a strength of at least 1 Tesla in at least half the volume of blood.

In some instances of the third method, the alternating magnetic field is applied to the subject's body for at least 12 hours per day, at least 5 days per week.

Another aspect of the invention is directed to a second apparatus for treating a liquid tumor in a subject's body that includes a volume of blood. The second apparatus comprises a platform, at least one alternating voltage source, and at least one electromagnetic coil. The at least one alternating voltage source has a frequency between 50 kHz and 1 MHz. The at least one electromagnetic coil is configured to generate at least one alternating magnetic field when driven by the at least one alternating voltage source. And the platform, the at least one alternating voltage source, and the at least one electromagnetic coil are configured and positioned such that when the subject's body is supported by the platform, the at least one alternating magnetic field generated by the at least one electromagnetic coil has a strength of at least 1 Tesla in at least one-third of the volume of blood.

In some embodiments of the second apparatus, the platform, the at least one alternating voltage source, and the at least one electromagnetic coil are configured and positioned such that when the subject's body is supported by the platform, the at least one alternating magnetic field generated by the at least one electromagnetic coil has a strength of at least 1 Tesla in at least half of the subject's ribs, at least half of the subject's vertebrae, at least half of the subject's sternum, and at least half the subject's pelvic bones. Optionally, in these embodiments, the platform, the at least one alternating voltage source, and the at least one electromagnetic coil are configured and positioned such that when the subject's body is supported by the platform, the at least one alternating magnetic field generated by the at least one electromagnetic coil has a strength of at least 1 Tesla in at least half the volume of blood.

In any of the embodiments or instances described above, the at least one alternating voltage source could optionally have a frequency between 50 kHz and 500 kHz, or between 80 kHz and 300 kHz.

### BRIEF DESCRIPTION OF THE DRAWINGS

FIG. 1 depicts a modified MRI machine that has the ability to apply alternating magnetic fields to a subject's body in addition to performing its conventional imaging function.

FIG. 2 depicts a flowchart showing how the modified MRI machine depicted in FIG. 1 can be used in both a first mode to perform imaging, and in a second mode to treat a subject using alternating magnetic fields.

FIG. 3 depicts a first embodiment for applying TTFields to larger sections of a subject's body.

FIGS. 4A and 4B respectively depict plan and side views of a second embodiment for applying TTFields to larger sections of a subject's body.

FIG. 5 depicts a third embodiment for applying TTFields to larger sections of a subject's body.

Various embodiments are described in detail below with reference to the accompanying drawings, wherein like reference numerals represent like elements.

### DESCRIPTION OF THE PREFERRED EMBODIMENTS

### Section 1

Alternating electric fields can be induced in a target region in a subject's body without relying on electrode assemblies positioned on the subject's body. More specifically, because an electric field and a magnetic field are interrelated aspects of the same underlying physical phenomena, an alternating electric field can be induced in a target region within a subject's body by applying an alternating *magnetic* field to the subject's body using one or more electromagnetic coils. And notably, these electromagnetic coils can be positioned outside the subject's body, and do not have to contact the subject's body in order to work.

There are, however, some hurdles associated with using an alternating magnetic field to induce an electric field in a target region in a subject's body. More specifically, inducing a 1 V/cm alternating electric field (i.e., the minimum field strength required to treat a tumor) by generating an alternating magnetic field within a subject's body requires hardware that is much heavier (e.g., >20 kg), much bulkier (e.g., >20 liters), much more power-hungry, and much more expensive than the prior art approach that relies on electrode assemblies positioned on the subject's skin. Taken together, these factors make it unlikely that magnetic field-based systems for generating alternating electric fields will be mass-produced to the point where they can be installed in the home of every person who needs alternating electric field therapy.

The embodiments described in this section below take the opposite approach to in-home installations. More specifically, instead of attempting to install a heavy, bulky, power-hungry, and expensive system in every person's home who needs alternating electric field therapy, these embodiments rely on conventional magnetic resonance imaging (MRI) machines that have been modified as described herein to generate alternating magnetic fields. These modified MRI machines are referred to herein as MRI+ machines. Each patient in need of alternating electric field therapy makes a visit to an MRI center and uses a respective MRI+ machine. The MRI+ machine will generate an alternating magnetic field, which will in turn induce an alternating electric field (e.g., with an amplitude of 1-10 V/cm) within the subject's body. Based on the proven efficacy of TTFields, this alternating electric field will provide the desired therapeutic effect (e.g., treating a tumor or increasing the permeability of the subject's blood brain barrier).

The MRI+ machine can scan for images, e.g. of a tumor, and can also deliver an alternating electromagnetic field to the tumor at a frequency and intensity designed to kill the tumor. The obstacles to delivering adequate electric field intensity via surface electrodes to an internal target are overcome using a magnetic approach. The relative magnetic permeability of all biological tissues is 1, so magnetic field strength is proportional to the distance between the magnetic coils and the target and the field is relatively uniform. In other words, the magnetic field is neither impeded by resistive structures nor shunted by conductive structures. Brain cancer patients will not need to shave their heads to receive magnetic TTFields, and the magnetic coils in an MRI machine do not cause skin irritation or other side effects.

The magnetic field in an MRI machine can be directed via one or more of the various types of coils (e.g. fixed-field, gradient, RF, surface) to a given target, and just as imaging of large parts of the body can be achieved, so can delivery of alternating magnetic fields to liquid and dispersed tumors be achieved, so that one location of the tumor is not treated at the expense of the other locations growing or metastasizing.

Notably, conventional MRI machines are typically only used during business hours to perform their conventional imaging function, and are not often used at night. Accordingly, if a conventional MRI machine is modified to become an MRI+ machine, the resulting MRI+ machine can be used to perform MRI imaging during business hours, and can also be used to apply alternating electric fields to subjects in need thereof at night, thereby increasing the overall utilization of the machine. And because the vast majority of the hardware that is needed to implement an MRI+ machine is already present in all conventional MRI machines, an MRI+ machine can be built by retrofitting a conventional MRI machine with a software update and a relatively small hardware modification.

Conventional MRI machines all have a static magnetic field electromagnet and also have a plurality of electromagnetic coils including radiofrequency transmitter and receiver coils, gradient coils, and, optionally, surface coils. The orientation and position of the radiofrequency transmitter coils in conventional MRI machines is such that energizing a first subset of these coils with an AC signal will create an alternating magnetic field in the subject's body in a first direction, and that energizing a second subset of these coils with an AC signal will create an alternating magnetic field in the subject's body in a second direction that is roughly perpendicular to the first direction, since the change of direction of the applied field has been shown to increase the efficacy of TTFields. (This, of course, assumes that the target region of the subject's body is positioned in the core of the conventional MRI machine.)

Conventional MRI machines also have a radiofrequency amplifier/transmitter (RA/T) that drives the radiofrequency transmitter coils, and a controller that instructs the RA/T to energize the radiofrequency transmitter coils. Assume, for example, that we want to treat a given subject with alternating electric fields at 200 kHz. If the RA/T in a given conventional MRI machine is capable of driving the radiofrequency transmitter coils at 200 kHz, no hardware modifications to the conventional MRI machine will be required other than reprogramming of the controller to initiate the functions described herein. If, on the other hand, the RA/T in a given conventional MRI machine is not capable of driving the radiofrequency transmitter coils at 200 kHz, the RA/T of that MRI machine should be replaced with a substitute RA/T that can operate at all the original frequencies as well as 200 kHz, and the controller should also be reprogrammed. In either case, when the RA/T drives the radiofrequency transmitter coils with a 200 kHz signal (in response to instructions received from the reprogrammed controller), the radiofrequency transmitter coils will generate a 200 kHz magnetic field in the subject's body. And this 200 kHz magnetic field will induce a 200 kHz electric field that will provide the desired therapeutic effect.

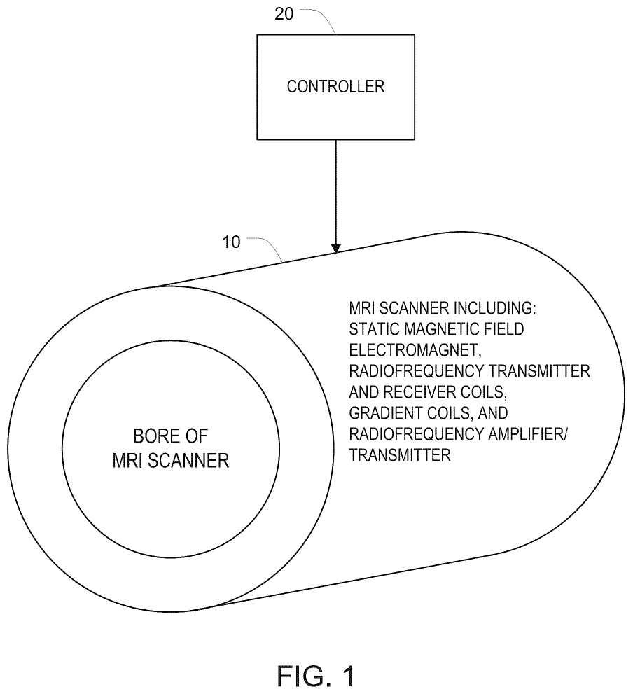

FIG. 1 depicts an MRI+ machine 10 that is based on a conventional MRI machine, modified as described herein. Like all conventional MRI machines, it has an electromagnet configured to generate a static magnetic field within a bore and a plurality of electromagnetic coils including (a) a set of radiofrequency transmitter and receiver coils configured to generate and receive electromagnetic fields within the static magnetic field; (b) a plurality of gradient coils configured to introduce magnetic gradients superimposed upon the static magnetic field; and, optionally, (c) a plurality of surface coils. The MRI+ machine 10 also has a radiofrequency amplifier/transmitter that drives the radiofrequency transmitter coils.

The MRI+ machine 10 has a controller 20 configured to operate the MRI scanner in a first mode in which images of a body part positioned within the bore are obtained, just as in a conventional MRI machine. But unlike conventional MRI machines, the controller 20 is further configured to operate the MRI scanner in a second mode, in order to provide a desired therapeutic effect. The second mode can be either a bidirectional second mode or a unidirectional second mode.

In those embodiments where the second mode is bidirectional, the controller 20 issues commands to the RA/T that cause the RA/T to (a) drive a first subset of the radiofrequency transmitter coils with an AC current at a frequency between 5 kHz and 1 MHz (e.g., between 100 kHz and 300 kHz) for a first interval of time so that a first magnetic field is induced in the body part; and (b) drive a second subset of the radiofrequency transmitter coils with an AC current at a frequency between 5 kHz and 1 MHz (e.g., between 100 kHz and 300 kHz) for a second interval of time so that a second magnetic field is induced in the body part. Because the first and second subsets of the radiofrequency transmitter coils have different positions in space, the first magnetic field and the second magnetic field have different directions. For example, if the first subset of the radiofrequency transmitter coils are mounted perpendicularly with respect to the second subset of the radiofrequency transmitter coils, the first magnetic field and the second magnetic field will be perpendicular.

The two driving steps (a) and (b) described in the previous paragraph are repeated in an alternating sequence a plurality of times (e.g., at least 100 times). The controller 20 continues to issue commands to the RA/T so that the sum of all the first intervals of time is at least one hour, and the sum of all the second intervals of time is at least one hour. For example, if each first interval of time is one minute long and each second interval of time is one minute long, and steps (a) and (b) are repeated in an alternating sequence 100 times, the sum of all the first intervals of time will be 100 minutes, and the sum of all the second intervals of time will be 100 minutes, which taken together add up to 200 minutes.

In some embodiments, each of the first intervals of time is between 25 ms and 1800 s, and each of the second intervals of time is between 25 ms and 1800 s. In some embodiments, each of the first intervals of time is between 0.2 s and 60 s, and each of the second intervals of time is between 0.2 and 60 s.

Note that in some embodiments, the large and strong electromagnet that is configured to generate the static magnetic field within the bore remains active while the MRI scanner is operating in the second mode. But in alternative embodiments, that electromagnet remains deactivated while the MRI scanner is operating in the second mode. In either case, the radiofrequency transmitter coils will induce the relevant magnetic fields in the body part.

In those embodiments where the second mode is unidirectional, the controller 20 issues commands to the RA/T that cause the RA/T to drive a subset of the radiofrequency transmitter coils with an AC current at a frequency between 5 kHz and 1 MHz (e.g., between 100 kHz and 300 kHz) so that an alternating magnetic field is induced in the body part, and the driving continues for at least four hours.

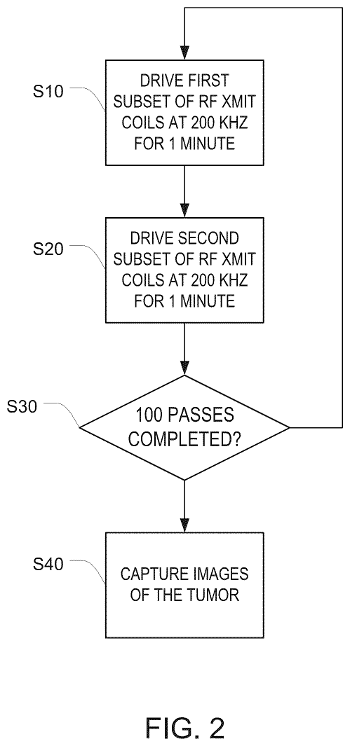

FIG. 2 depicts a flowchart showing how an MRI+ scanner can be used in both the first mode to perform imaging, and in a bidirectional second mode to treat a subject using alternating electric fields (e.g., TTFields). In this example, steps S10-S30 correspond to the bidirectional second mode, and step S40 corresponds to the first mode (i.e., the imaging mode).

The method of FIG. 2 begins in step S10, where a first subset of the radiofrequency transmitter coils are driven with an AC current at a frequency between 5 kHz and 1 MHz (e.g., 100 kHz-300 kHz) for a first interval of time so that a first alternating magnetic field is induced in the body part. Next, in step S20, a second subset of the radiofrequency transmitter coils are driven with an AC current at a frequency between 5 kHz and 1 MHz (e.g., 100 kHz-300 kHz) for a second interval of time so that a second alternating magnetic field is induced in the body part. The first alternating magnetic field and the second alternating magnetic field have different directions. In step S30, a test is performed to ascertain whether 100 passes through the loop have been completed. If 100 passes have not been completed, processing returns to step S10. But if 100 passes through the loop have been completed, processing proceeds to step S40.

Each of the intervals of time described in the previous paragraph (i.e., the first intervals of time and the second intervals of time) could be between 25 ms and 1800 s, or between 0.2 s and 60 s. The durations of these intervals should be selected so that a sum of all the first intervals of time is at least one hour, and the sum of all the second intervals of time is at least one hour.

Step S40 is where the MRI+ scanner is used to capture an image of whatever happens to be positioned within the bore of the scanner. This could be, for example, a tumor in the body part that is positioned within the bore of the scanner. This is accomplished in the same way that a conventional MRI scanner performs imaging.

Note that the embodiments described above in connection with FIGS. 1 and 2 all rely on the radiofrequency transmitter coils in the second operating mode in order to provide the desired therapeutic effect. But in alternative embodiments, the system could instead drive one of the other types of electromagnetic coils contained within the MRI machine (e.g., the gradient coils or the surface coils) to provide a similar therapeutic effect.

Advantageously, the embodiments described above extend the capabilities of conventional MRI machines to provide a new functionality above and beyond capturing images. More specifically, the MRI+ machines described above can be used to both capture images and provide a therapeutic effect by inducing alternating magnetic fields in portions of the subject's body. And because electric fields and magnetic fields are interrelated aspects of the same underlying physical phenomena, the alternating magnetic field will induce a corresponding alternating electric field, which will, in turn, produce the desired therapeutic effect. These therapeutic effects include, but are not limited to treating a tumor using TTFields, or increasing the permeability of the blood brain barrier (e.g., to help a chemotherapy drug reach the subject's brain).

### Section 2

When TTFields are applied to a localized, relatively small region of a subject's body (e.g., a section that includes less than 10% or less than 20% of the subject's blood), cancerous cells that lie outside of this small section will not be affected by the TTFields. And because liquid tumors (e.g., leukemia, lymphoma, and myeloma) circulate throughout the body in the blood, applying TTFields to a section that includes less than 20% of the subject's blood means that any cancer cells that reside in the remaining >80% of the subject's blood are not being targeted at any given moment in time.

On the other hand, if TTFields can be applied to a much larger section of the subject's body (e.g., a section that includes at least ¾, at least ⅔, or at least half of the subject's blood), the TTFields should be able to target the majority of cancer cells that reside in the subject's body at any given moment in time. It may even be sufficient to apply TTFields to at least ⅓ of the subject's blood. This section describes using one or more electromagnetic coils to apply an alternating magnetic field to very large portions of the subject's body (e.g., portions that include at least half the subject's blood). The alternating magnetic field induces an alternating electric field in those same very large portions, which makes it practical to treat liquid tumors using TTFields.

A first approach for applying TTFields to larger sections of the subject's body (e.g., a section that includes at least half of the subject's blood) is to position transducer arrays over the entire front, rear, left, and right surfaces of the subject's torso. Those transducer arrays can then be used to induce TTFields in a manner similar to the way the NovoTTF-100L system treats malignant pleural mesothelioma, except using much larger transducer arrays. While this arrangement is workable, some subjects may find the very large transducer arrays to be uncomfortable and/or limit their mobility.

A second approach for applying TTFields to larger sections of the subject's body (e.g., a section that includes at least half of the subject's blood) is to apply an alternating *magnetic* field to a section of the subject's body that includes e.g., at least half of the subject's blood. This alternating magnetic field will in turn induce an alternating electric field in that same section of the subject's body. The alternating magnetic field has a frequency between 50 kHz and 1 MHz (e.g., 50 kHz-500 kHz, 80 kHz-300 kHz, or 100 kHz-250 kHz) and is configured so that the alternating electric field that is induced by the magnetic fields will have a strength of at least 0.1 V/cm (or, more preferably, at least 1 V/cm) in at least ⅓ of the volume of the blood in the subject's body, or more preferably at least ½, at least ⅔, or at least ¾ of the volume of the blood in the subject's body.

This can be accomplished, for example, by driving at least one electromagnetic coil with at least one alternating voltage source at a frequency between 50 kHz and 1 MHz. The at least one electromagnetic coil is configured and positioned with respect to the subject's body so that when the electromagnetic coil is driven by the alternating voltage source, the electromagnetic coil will generate an alternating magnetic field. And the at least one electromagnetic coil is positioned with respect to the subject's body so that the at least one alternating magnetic field generated by the at least one electromagnetic coil will induce an electric field with a strength of at least 0.1 V/cm in at least one-third of the volume of blood contained in the subject's body.

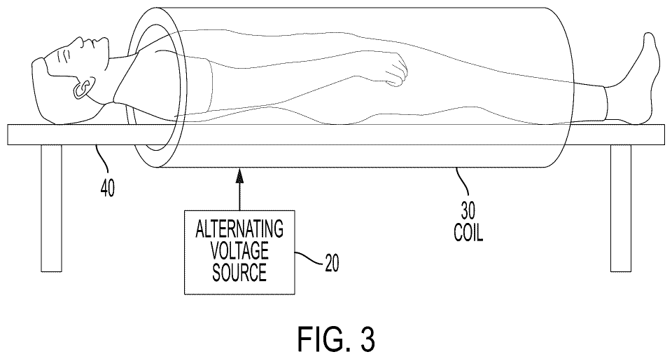

FIG. 3 depicts a first embodiment for implementing the second approach for applying TTFields to larger sections of the subject's body (e.g., sections that include at least ⅓ of the volume of the blood in the subject's body, or more preferably at least ½, at least ⅔, or at least ¾ of the volume of the blood in the subject's body). This embodiment uses a single large electromagnetic coil 30 that is large enough so that a person's body can fit in the void in the center of the electromagnetic coil 30. The person's body is supported by a platform 40 that is shaped and dimensioned to support the subject's body.

The electromagnetic coil 30 is positioned with respect to the platform 40 so that when the subject's body is supported by the platform 40 and an alternating voltage source 20 drives the electromagnetic coil 30, the electromagnetic coil 30 will generate an alternating magnetic field; and this alternating magnetic field will be applied to the subject's body. The alternating voltage source has a frequency between 50 kHz and 1 MHz, and as a result, the alternating magnetic field will also have a frequency between 50 kHz and 1 MHz. The nature of the alternating magnetic field generated by the single large electromagnetic coil 30 is such that it will induce an alternating electric field having a strength of at least 0.1 V/cm (and more preferably at least 1 V/cm or at least 10 V/cm) in at least one-third of the total volume of the blood contained in the subject's body.

The inner diameter of the electromagnetic coil 30 must be large enough so that the subject (e.g., a person) can fit within the void in the interior of the electromagnetic coil 30. And the length of the electromagnetic coil 30 is long enough so that at least ⅓ of the volume of the blood in the subject's body (or more preferably at least ½, at least ⅔, or at least ¾ of the volume of the blood in the subject's body) will fit inside the electromagnetic coil 30. Because a large portion of any given subject's blood resides in the subject's torso, this may be accomplished, for example, by making the electromagnetic coil 30 long enough so that the entire torso of the subject's body can fit inside the electromagnetic coil 30. Notably, when the electromagnetic coil is long enough for the subject's entire torso to fit inside the electromagnetic coil 30, the subject's ribs, lumbar vertebrae, thoracic vertebrae, sternum, and pelvic bones will all be positioned inside the electromagnetic coil 30. This is advantageous because those anatomical structures house a very large percentage of the subject's bone marrow, and liquid tumors reside in both the blood and bone marrow.

In some preferred embodiments, the nature of the alternating magnetic field generated by the single electromagnetic coil 30 is such that it will induce an alternating electric field having a strength of at least 1 V/cm in all of the subject's ribs, all of the subject's lumbar and thoracic vertebrae, the subject's entire sternum, and all of the subject's pelvic bones. These embodiments are particularly advantageous because most of the subject's blood and most of the subject's bone marrow will all be subjected to an alternating electric field at a field strength of at least 1 V/cm, which is beneficial for treating liquid tumors. But in other preferred embodiments (not shown), the electromagnetic coil 30 can be shorter, to an extent that the alternating magnetic field generated by the single electromagnetic coil 30 will only induce an alternating electric field having a strength of at least 1 V/cm in at least half of the subject's ribs, at least half of the subject's vertebrae, at least half of the subject's sternum, and at least half the subject's pelvic bones.

When treating liquid tumors using alternating electric fields at field strengths of at least 1 V/cm that are induced by alternating magnetic fields, it can be beneficial to apply the alternating magnetic field to the subject's body for at least 12 hours per day, at least five days per week. The induced electric field will inhibit the proliferation of liquid tumor cancer cells in the subject's body. For example, the induced electric field will reduce the number of liquid tumor cancer cells in the subject's body by at least 10%. When the induced electric field is stronger (i.e., at least 10 V/cm), the amount of time that the alternating magnetic field must be applied to the subject's body can be reduced e.g., to at least two hours per day, at least three days per week.

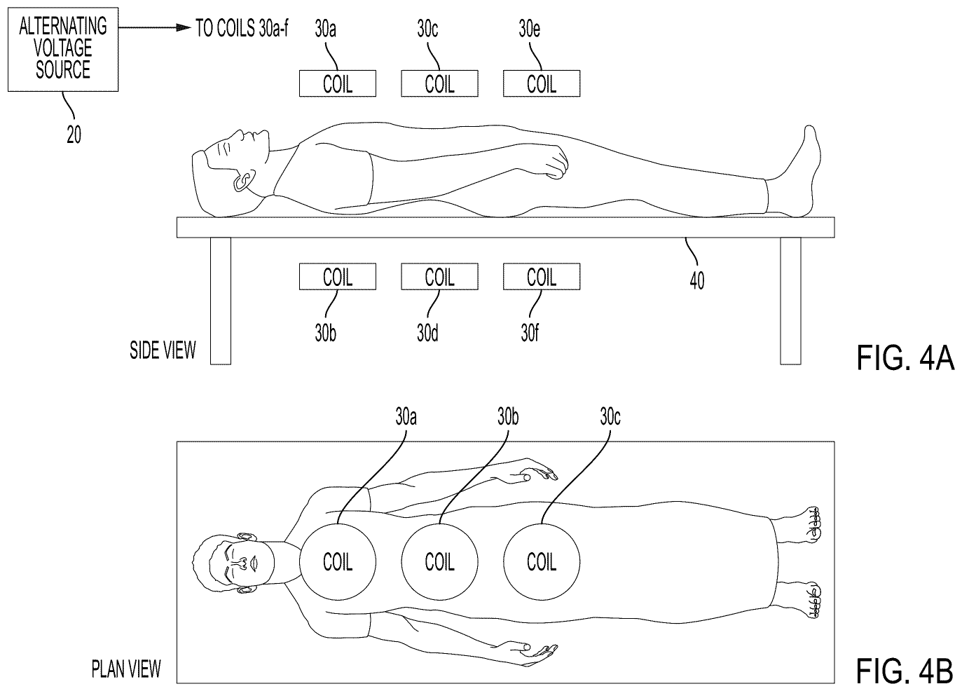

FIGS. 4A and 4B respectively depict plan and side views of a second embodiment for implementing the second approach for applying TTFields to larger sections of the subject's body (e.g., sections that include at least ⅓ of the volume of the blood in the subject's body, or more preferably at least ½, at least ⅔, or at least ¾ of the volume of the blood in the subject's body). This embodiment uses a plurality of electromagnetic coils 30a-f that are positioned in the vicinity of the subject's body. The person's body is supported by a platform 40 that is shaped and dimensioned to support the subject's body.

The electromagnetic coils 30a-f are positioned with respect to the platform 40 so that when the subject's body is supported by the platform 40 and an alternating voltage source 20 drives the electromagnetic coils 30a-f, the electromagnetic coils 30a-f will generate respective alternating magnetic fields; and these alternating magnetic fields will be applied to the subject's body. The alternating voltage source has a frequency between 50 kHz and 1 MHz, and as a result, the alternating magnetic fields will also have a frequency between 50 kHz and 1 MHz.

Note that in the embodiment depicted in FIG. 4A, a single alternating voltage source 20 drives all of the coils 30a-f, which may be accomplished, for example, by wiring all of those coils 30a-f in parallel. But in alternative embodiments, the coils 30a-f could be wired in series or in a series-parallel or parallel-series combination. In yet other alternative embodiments (not shown) each of the coils 30a-f could be driven by its own individual voltage source, or the coils 30a-f can be arranged in a plurality of groups, with each of the groups being driven by its own individual voltage source (e.g., with the coils within each group being wired in series or in parallel).

The nature of the alternating magnetic fields generated by the electromagnetic coils 30a-f are such that the alternating magnetic fields will induce corresponding alternating electric fields having strengths of at least 0.1 V/cm (and more preferably at least 1 V/cm or at least 10 V/cm) in at least one-third of the total volume of the blood contained in the subject's body.

The quantity, size, and position of the coils 30a-f must be such that the magnetic fields that emanate from the coils 30a-f will cover at least ⅓ of the volume of the blood in the subject's body (or more preferably at least ½, at least ⅔, or at least ¾ of the volume of the blood in the subject's body). Because a large portion of any given subject's blood resides in the subject's torso, this may be accomplished, for example, by making the quantity, size, and position of the electromagnetic coils 30a-f such that the entire torso of the subject's body will be covered by the magnetic fields that emanate from the coils 30a-f. Notably, in this situation, the subject's ribs, lumbar vertebrae, thoracic vertebrae, sternum, and pelvic bones will all be covered by the magnetic fields that emanate from the coils 30a-f.

In some preferred embodiments, the nature of the alternating magnetic fields generated by the electromagnetic coils 30a-f is such that it will induce an alternating electric field having a strength of at least 1 V/cm in all of the subject's ribs, all of the subject's lumbar and thoracic vertebrae, the subject's entire sternum, and all of the subject's pelvic bones. as noted above, this is particularly advantageous because most of the subject's blood and most of the subject's bone marrow will all be subjected to an alternating electric field at a field strength of at least 1 V/cm, which is beneficial for treating liquid tumors. But in other preferred embodiments (not shown), the coverage of the electromagnetic coils 30 a-f will only induce an alternating electric field having a strength of at least 1 V/cm in at least half of the subject's ribs, at least half of the subject's vertebrae, at least half of the subject's sternum, and at least half the subject's pelvic bones.

The recommended field strengths and minimum-time recommendations for the alternating electric fields that are induced by the alternating magnetic field in this FIG. 4A/B embodiment are similar to the recommended field strengths and minimum-time recommendations described above in connection with the FIG. 3 embodiment.

A third approach for applying TTFields to larger sections of the subject's body (e.g., a section that includes at least half of the subject's blood) is to apply an alternating *magnetic* field to a section of the subject's body that includes e.g., at least half of the subject's blood. The alternating magnetic field has a frequency between 50 kHz and 1 MHz (e.g., 50 kHz-500 kHz, 80 kHz-300 kHz, or 100 kHz-250 kHz) and a strength of at least 1 T in at least ⅓ of the volume of the blood in the subject's body, or more preferably at least ½, at least ⅔, or at least ¾ of the volume of the blood in the subject's body. The alternating magnetic field will in turn induce an alternating electric field (i.e., TTFields) in that same section of the subject's body.

This can be accomplished, for example, by driving at least one electromagnetic coil with at least one alternating voltage source at a frequency between 50 kHz and 1 MHz. The at least one electromagnetic coil is configured and positioned with respect to the subject's body so that when the electromagnetic coil is driven by the alternating voltage source, the electromagnetic coil will generate an alternating magnetic field with a strength of at least 1 T in at least one-third of the volume of blood contained in the subject's body.

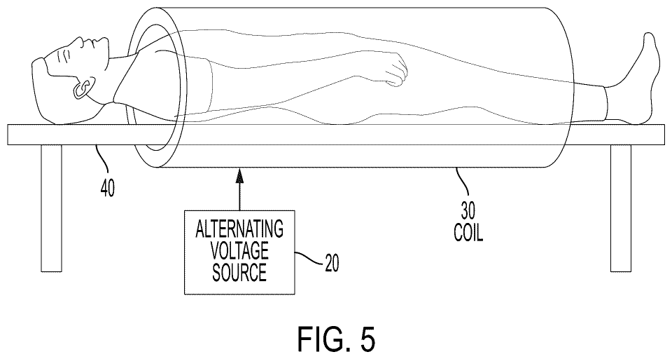

FIG. 5 depicts an embodiment for implementing the third approach for applying TTFields to larger sections of the subject's body (e.g., sections that include at least ⅓ of the volume of the blood in the subject's body, or more preferably at least ½, at least ⅔, or at least ¾ of the volume of the blood in the subject's body). This embodiment uses a single large electromagnetic coil 30 that is large enough so that a person's body can fit in the void in the center of the electromagnetic coil 30. The person's body is supported by a platform 40 that is shaped and dimensioned to support the subject's body.

The electromagnetic coil 30 is positioned with respect to the platform 40 so that when the subject's body is supported by the platform 40 and an alternating voltage source 20 drives the electromagnetic coil 30, the electromagnetic coil 30 will generate an alternating magnetic field; and this alternating magnetic field will be applied to the subject's body. The alternating voltage source has a frequency between 50 kHz and 1 MHz, and as a result, the alternating magnetic field will also have a frequency between 50 kHz and 1 MHz. The nature of the alternating magnetic field generated by the single large electromagnetic coil 30 is such that it has a strength of at least 1 T in at least one-third of the total volume of the blood contained in the subject's body. The alternating magnetic field will in turn induce an alternating electric field (i.e., TTFields) in the same section of the subject's body.

The inner diameter of the electromagnetic coil 30 must be large enough so that the subject (e.g., a person) can fit within the void in the interior of the electromagnetic coil 30. And the length of the electromagnetic coil 30 is long enough so that at least ⅓ of the volume of the blood in the subject's body (or more preferably at least ½, at least ⅔, or at least ¾ of the volume of the blood in the subject's body) will fit inside the electromagnetic coil 30. Because a large portion of any given subject's blood resides in the subject's torso, this may be accomplished, for example, by making the electromagnetic coil 30 long enough so that the entire torso of the subject's body can fit inside the electromagnetic coil 30. Notably, when the electromagnetic coil is long enough for the subject's entire torso to fit inside the electromagnetic coil 30, the subject's ribs, lumbar vertebrae, thoracic vertebrae, sternum, and pelvic bones will all be positioned inside the electromagnetic coil 30. As explained above, this is advantageous because those anatomical structures house a very large percentage of the subject's bone marrow, and liquid tumors reside in both the blood and bone marrow.

In some preferred embodiments, the nature of the alternating magnetic field generated by the single electromagnetic coil 30 is such that it has a strength of at least 1 T in all of the subject's ribs, all of the subject's lumbar and thoracic vertebrae, the subject's entire sternum, and all of the subject's pelvic bones. These embodiments are particularly advantageous because most of the subject's blood and most of the subject's bone marrow will all be subjected to an alternating magnetic field of at least 1 T, which is beneficial for treating liquid tumors. But in other preferred embodiments (not shown), the electromagnetic coil 30 can be shorter, to an extent that the alternating magnetic field generated by the single electromagnetic coil 30 will only induce an alternating magnetic field of at least 1 T in at least half of the subject's ribs, at least half of the subject's vertebrae, at least half of the subject's sternum, and at least half the subject's pelvic bones.

When treating liquid tumors using alternating magnetic fields at field strengths of at least 1 T, the treatment should be applied to the subject's body for at least two hours per day, at least three days per week. In some preferred embodiments, the alternating magnetic field is applied to the subject's body for at least 12 hours per day, at least five days per week. The alternating magnetic field will inhibit the proliferation of liquid tumor cancer cells in the subject's body. For example, the alternating magnetic field will reduce the number of liquid tumor cancer cells in the subject's body by at least 10%.

The embodiment described above in connection with FIGS. 4A and 4B can also be used to implement the third approach for applying TTFields to larger sections of the subject's body by applying an alternating magnetic field to the subject's body at a frequency between 50 kHz and 1 MHz with a field strength of at least 1 T in at least ⅓ of the volume of the blood in the subject's body (or more preferably at least ½, at least ⅔, or at least ¾ of the volume of the blood in the subject's body). In some preferred embodiments, the 1 T alternating magnetic field is applied for at least two hours per day, at least three days per week.

Notably, in any of the embodiments described in this section above, the frequency range of the alternating magnetic field can be narrower (e.g., 50 kHz-500 kHz, 80 kHz-300 kHz, or 100 kHz-250 kHz). In these embodiments, the alternating electric field that is induced by the alternating magnetic field will have a respective corresponding frequency.

### CONCLUSION

The methods described herein can be implemented, in whole or in part, in software that can be stored in computer-readable media for execution by one or more computer processors. For example, the computer-readable media can be volatile memory (e.g., RAM) non-volatile memory (e.g., ROM, PROM, EPROM, solid state drives, hard drives, etc.). Additionally or alternatively, the methods described herein can be implemented in computer hardware including but not limited to one or more application-specific integrated circuits (ASICs).

Finally, it is important to note that the usage of the identifiers (a), (b), (c), etc. in the claims below does not imply a particular sequence in time for the corresponding steps. For while it is certainly possible that step (a) will precede step (b) in time, different sequencings of those steps are also possible, except in cases where a particular sequencing is inconsistent with the internal language of the various steps or with other language in the claims. For example, a step labeled (b) could precede a step labeled (a) in time. It is also possible for two or more steps to occur simultaneously or to overlap to an extent, except in cases where simultaneity or overlapping would be inconsistent with the internal language of the various steps or with other language in the claims.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An MRI scanner comprising:
an electromagnet configured to generate a static magnetic field within a bore;
a plurality of electromagnetic coils including a set of radiofrequency transmitter and receiver coils configured to generate and receive electromagnetic fields within the static magnetic field, a plurality of gradient coils configured to introduce magnetic gradients superimposed upon the static magnetic field, and, optionally, a plurality of surface coils; and
a controller,
wherein the controller is configured to operate the MRI scanner in a first mode in which images of a body part positioned within the bore are obtained,
wherein the controller is further configured to operate the MRI scanner in a second mode in which
(a) a first subset of the electromagnetic coils is driven with an AC current at a frequency between 5 kHz and 1 MHz for a first interval of time so that a first alternating magnetic field is induced in the body part,
(b) a second subset of the electromagnetic coils is driven with an AC current at a frequency between 5 kHz and 1 MHz for a second interval of time so that a second alternating magnetic field is induced in the body part, wherein the first alternating magnetic field and the second alternating magnetic field have different directions, and
(c) steps (a) and (b) are repeated in an alternating sequence a plurality of times,
wherein a sum of all the first intervals of time is at least one hour, and
wherein a sum of all the second intervals of time is at least one hour.

2. The MRI scanner of claim 1, wherein the electromagnetic coils that are driven with the AC current are radiofrequency transmitter coils.

3. The MRI scanner of claim 1, wherein the electromagnetic coils that are driven with the AC current are gradient coils.

4. The MRI scanner of claim 1, wherein the electromagnetic coils that are driven with the AC current are surface coils.

5. The MRI scanner of claim 1, wherein the first subset of the electromagnetic coils is driven with an AC current at a frequency between 100 kHz and 300 kHz, and wherein the second subset of the electromagnetic coils is driven with an AC current at a frequency between 100 kHz and 300 kHz.

6. An MRI scanner comprising:
an electromagnet configured to generate a static magnetic field within a bore;
a plurality of electromagnetic coils including a set of radiofrequency transmitter and receiver coils configured to generate and receive electromagnetic fields within the static magnetic field, a plurality of gradient coils configured to introduce magnetic gradients superimposed upon the static magnetic field, and, optionally, a plurality of surface coils; and
a controller,
wherein the controller is configured to operate the MRI scanner in a first mode in which images of a body part positioned within the bore are obtained, and
wherein the controller is further configured to operate the MRI scanner in a second mode in which a subset of the electromagnetic coils is driven with an AC current at a frequency between 5 kHz and 1 MHz so that an alternating magnetic field is induced in the body part for at least four hours.

7. The MRI scanner of claim 6, wherein the electromagnetic coils that are driven with the AC current are radiofrequency transmitter coils.

8. The MRI scanner of claim 6, wherein the electromagnetic coils that are driven with the AC current are gradient coils.

9. The MRI scanner of claim 6, wherein the electromagnetic coils that are driven with the AC current are surface coils.

10. The MRI scanner of claim 6, wherein the subset of the electromagnetic coils is driven with an AC current at a frequency between 100 kHz and 300 kHz.

11. A method of treating a liquid tumor in a subject's body that includes a volume of blood, the method comprising:
applying an alternating magnetic field to the subject's body,
wherein the alternating magnetic field has a frequency between 50 kHz and 1 MHz, and
wherein the alternating magnetic field induces an alternating electric field having a strength of at least 0.1 V/cm in at least one-third of the volume of blood.

12. The method of claim 11, wherein the induced alternating electric field inhibits proliferation of liquid tumor cancer cells in the subject's body.

13. The method of claim 11, wherein the alternating magnetic field induces an alternating electric field having a strength of at least 1 V/cm in at least one-third of the volume of blood.

14. The method of claim 13, wherein the alternating magnetic field is applied to the subject's body for at least 12 hours per day, at least 5 days per week.

15. The method of claim 14, wherein the induced alternating electric field has a strength of at least 1 V/cm in at least half of the subject's ribs, at least half of the subject's vertebrae, at least half of the subject's sternum, and at least half the subject's pelvic bones.

16. The method of claim 11, wherein the alternating magnetic field induces an alternating electric field having a strength of at least 10 V/cm in at least one-third of the volume of blood.

17. The method of claim 16, wherein the alternating magnetic field is applied to the subject's body for at least 2 hours per day, at least 3 days per week.

18. The method of claim 17, wherein the induced alternating electric field has a strength of at least 10 V/cm in at least half of the subject's ribs, at least half of the subject's vertebrae, at least half of the subject's sternum, and at least half the subject's pelvic bones.

19. The method of claim 18, wherein the alternating magnetic field has a frequency between 80 kHz and 300 kHz.

20. The method of claim 11, wherein the alternating magnetic field has a frequency between 80 kHz and 300 kHz.
